SEQUENCE LISTING (27 sequences)

SEQ ID NO: 1 - DNA, 2880 bp, Homo sapiens
gatggctggggctcttgagctataagtggcacctcagggccctgacgggcgtctcgccatgctgctcctgggcctgctgctgctgcccctgctggctggcgcccgcctgctgtggaactggtggaagctccggagcctccacctcccgcctcttgccccgggcttcttgcacttgctgcagcccgacctcccaatctatctgcttggcctgactcagaaattcgggcccatctacaggctccaccttgggctgcaaggtgagaggctgatctcgctctggccctcaccataggagggggcggaggtgacggagagggtcctctctccgctgacgctgctttggctgtctcccagatgtggtggtgctgaactccaagaggaccattgaggaagccatggtcaaaaagtgggcagactttgctggcagacctgagccacttacctgtaagggctgggggcattttttctttcttaaacaaatttttttttaagagatgggttcttgctatgttgcccaggctggtcttaaattcctagtctcaaatgatcctcccacctcagcctcaagtgtgagccacctttggggcatccccaatccaggtccctggaagctcttggggggcatatctggtggggagaaagcaggggttggggaggccgaagaaggtcaggccctcagctgccttcatcagttcccaccctccagcccccacctcctcctgcagacaagctggtgtctaagaactacccggacctgtccttgggagactactccctgctctggaaagcccacaagaagctcacccgctcagccctgctgctgggcatccgtgactccatggagccagtggtggagcagctgacccaggagttctgtgaggtaaggctgggctcctgaggccacctcgggtcagcctcgcctctcacagtagcccccgccctgccgctgcacagcggcctgctgaactcacactgtttctccacagcgcatgagagcccagcccggcacccctgtggccattgaggaggaattctctctcctcacctgcagcatcatctgttacctcaccttcggagacaagatcaaggtgcctcacagcccctcaggcccacccccagcccctccctgagcctctccttgtcctgaactgaaagtactccctccttttctggcaggacgacaacttaatgcctgcctattacaaatgtatccaggaggtgttaaaaacctggagccactggtccatccaaattgtggacgtgattccctttctcagggtgaggacctggagcctagacacccctgggttgtaggggagaggctggggtggagggagaggctccttcccacagctgcattctcatgcttcctgccgcagttcttccccaatccaggtctccggaggctgaagcaggccatagagaagagggatcacatcgtggagatgcagctgaggcagcacaaggtggggactgtacgtggacggcctcccctcggcccacagccagtgatgctaccggcctcagcattgctatgaggcgggttcttttgcataccccagttatgggcctgttgccactctgtactcctctccccaggccagccgctcagcccgctcctttcaccctctgcaggagagcctcgtggcaggccagtggagggacatgatggactacatgctccaaggggtggcgcagccgagcatggaagagggctctggacagctcctggaagggcacgtgcacatggctgcagtggacctcctgatcggtggcactgagaccacagcaaacaccctctcctgggccgtggtttttttgcttcaccaccctgaggtgcgtcctggggacaagcaaaaggctccttcccagcaacctggccagggcggtgggcaccctcactcagctctgagcactgtgcggctggggctgtgcttgcctcaccggcactcaggctcactgggttgctgagggagcggctggaggctgggcagctgtgggctgctggggcaggactccacccgatcattccccagattcagcagcgactgcaggaggagctagaccacgaactgggccctggtgcctccagctcccgggtcccctacaaggaccgtgcacggctgcccttgctcaatgccaccatcgccgaggtgctgcgcctgcggcccgttgtgcccttagccttgccccaccgcaccacacggcccagcaggtgactcccgagggttggggatgagtgaggaaagcccgagcccagggaggtcctggccagcctctaactccagcccccttcagcatctccggctacgacatccctgagggcacagtcatcattccgaacctccaaggcgcccacctggatgagacggtctgggagaggccacatgagttctggcctggtatgtgggggccgggggcctgccgtcaaaatgtggtggaggctggtccccgctgccgctgaacgcctccccacccacctgtccacccgcccgcagatcgcttcctggagccaggcaagaactccagagctctggccttcggctgcggtgccccggtgtgcctgggcgagccgctggcgcgcctggacctcttcgtggtgctgacccgactgctgcaggccttcacgctgctgccctccggggacgccctgccctccctgcagcccctgccccactgcagtgtcatcctcaagatgcagcctttccaagtgcggctgcagccccgggggatgggggcccacagcccaggccagaaccagtgatggggcaggaccgatgccagccgggtacctcagtttctcctttattgctcccgtacgaacccctcccctcccccctgtaaacacagtgctgcgagatcgctggcagagaaggcttc

SEQ ID NO: 2 - DNA, 21 bp, Artificial Sequence
  Description of Artificial Sequence PCR primer P1
tggaactggtggaagctccgg

SEQ ID NO: 3 - DNA, 17 bp, Artificial Sequence
  Description of Artificial Sequence PCR primer P2
gcatctccacgatgtga

SEQ ID NO: 4 - DNA, 23 bp, Artificial Sequence
  Description of Artificial Sequence PCR primer E1TS
gcgcccgcctgctgtggaactgg

SEQ ID NO: 5 - DNA, 23 bp, Artificial Sequence
  Description of Artificial Sequence PCR primer E1TAS
ggaggtcgggctgcagcaagtgc

SEQ ID NO: 6 - DNA, 18 bp, Artificial Sequence
  Description of Artificial Sequence PCR primer I2TS
gaagaaggtcaggccctc

SEQ ID NO: 7 - DNA, 18 bp, Artificial Sequence
  Description of Artificial Sequence PCR primer I2TAS
caaggacaggtccgggta

SEQ ID NO: 8 - DNA, 21 bp, Artificial Sequence
  Description of Artificial Sequence PCR primer P3
ttgtccttgggagactactcc

SEQ ID NO: 9 - DNA, 23 bp, Artificial Sequence
  Description of Artificial Sequence PCR primer P42
acctctcgcaccccagtatgact

SEQ ID NO: 10 - DNA, 23 bp, Artificial Sequence
  Description of Artificial Sequence PCR primer E4TS
cggcacccctgtggccattgagg

SEQ ID NO: 11 - DNA, 23 bp, Artificial Sequence
  Description of Artificial Sequence PCR primer E4TAS
gtgggcctgaggggctgtgaggc

SEQ ID NO: 12 - DNA, 24 bp, Artificial Sequence
  Description of Artificial Sequence Sense insert of pCAH-I2N
attaagcccccaactcctcctgca

SEQ ID NO: 13 - DNA, 25 bp, Artificial Sequence
  Description of Artificial Sequence Antisense insert of pCAH-I2N
taattgcaggaggagttggggggct

SEQ ID NO: 14 - DNA, 24 bp, Artificial Sequence
  Description of Artificial Sequence Sense insert of pCAH-I2N2
attaagcccccacctcctcctgca

SEQ ID NO: 15 - DNA, 24 bp, Artificial Sequence
  Description of Artificial Sequence Antisense insert of pCAH-I2N2
taattgcaggattaggtgggggct

SEQ ID NO: 16 - DNA, 24 bp, Artificial Sequence
  Description of Artificial Sequence Sense insert of pCAH-I2P
attaagcccccagctcctcctgca

SEQ ID NO: 17 - DNA, 24 bp, Artificial Sequence
  Description of Artificial Sequence Antisense insert of pCAH-I2P
taattgcaggaggagctgggggct

SEQ ID NO: 18 - DNA, 26 bp, Artificial Sequence
  Description of Artificial Sequence Sense insert of pCAH-E1N
cggccacctcccgcctctggccacat

SEQ ID NO: 19 - DNA, 26 bp, Artificial Sequence
  Description of Artificial Sequence Antisense insert of pCAH-E1N
tggccagaggcgggaggtggccgatg

SEQ ID NO: 20 - DNA, 26 bp, Artificial Sequence
  Description of Artificial Sequence Sense insert of pCAH-E1P
cggccacctcctgcctctggccacat

SEQ ID NO: 21 - DNA, 26 bp, Artificial Sequence
  Description of Artificial Sequence Antisense insert of pCAH-E1P
tggccagaggcaggaggtggccgatg

SEQ ID NO: 22 - DNA, 21 bp, Artificial Sequence
  Description of Artificial Sequence Sense insert of pCAH-E4N
attacagcatcatctgttacc

SEQ ID NO: 23 - DNA, 21 bp, Artificial Sequence
  Description of Artificial Sequence Antisense insert of pCAH-E4N
taatggtaacagatgatgctg

SEQ ID NO: 24 - DNA, 21 bp, Artificial Sequence
  Description of Artificial Sequence Sense insert of pCAH-E4P
attacagcatcaactgttacc

SEQ ID NO: 25 - DNA, 21 bp, Artificial Sequence
  Description of Artificial Sequence Antisense insert of pCAH-E4P
taatggtaacagttgatgctg

SEQ ID NO: 26 - DNA, 20 bp, Artificial Sequence
  Description of Artificial Sequence PCR primer
aattaaccctcactaaaggg

SEQ ID NO: 27 - DNA, 22 bp, Artificial Sequence
  Description of Artificial Sequence PCR primer
gtaatacgactcactatagggc

(19) United States
(12) Patent Application Publication
(10) Pub. No.: US 2003/0073082 A1
(43) Pub. Date: Apr. 17, 2003

(54) Diagnostic method and oligonucleotide chip for congenital adrenal hyperplasia

(76) Inventor: Dong Kyu Jin, Seoul (KR)

Correspondence Address: JACOBSON HOLMAN PLLC, 400 SEVENTH STREET N.W., SUITE 600, WASHINGTON, DC, 20004, US

(21) Appl. No.: 09/951,502

(22) Filed: Sep. 14, 2001

(51) Int. Cl. C12Q 1/68; C12P 19/34; B05D 3/00

(52) U.S. Cl. 435/6; 435/91.2; 427/2.11

(57) ABSTRACT

The present invention provides a diagnostic method for congenital adrenal hyperplasia, comprising the steps of: preparing oligonucleotide chip in which 7 sets of tandem cDNA fragments of normal and patient's 21-hydroxylase gene (exon 1, intron 2, exon 4) are immobilized to solid support; amplifying the DNA segments of 21-hydroxylase gene (exon 1, intron 2, exon 4) from the genomnic DNA of a testee using labeled primers under the suitable condition for carrying out polymerase chain reaction (PCR); hybridizing 7 sets of tandem cDNA fragments in oligonucleotide chip with the PCR product by amplifying gDNA segments; and analyzing the results of the hybridization.

### BACKGROUND OF THE INVENTION

The present invention relates to a diagnostic method and oligonucleotide chip for congenital adrenal hyperplasia. More particularly, the present invention relates to using gene amplification methodology (Polymerase Chain Reaction), PCR-MPH (Polymerase Chain Reaction—Microtiter Plate Hybridization) and oligonucleotide probe technology. The methods and probes of the invention specifically relate to the detection of congenital adrenal hyperplasia. The invention relates to the fields of molecular biology, diagnostic medicine.

Congenital adrenal hyperplasia (CAH) is an inherited disorder of cortisol biosynthesis. Although five different enzymes, such as, StAR protein (Steroidogenic Acute Regulatory protein), 17-hydroxylase, 3β-HSD, 21-hydroxylase, 11β-hydroxylase are required to synthesize cortisol in the adrenal cortex-mix, steroid 21-hydroxylase deficiency accounts for more than 90% of CAH. The patient of CAH shows various pathogenic symptom due to the reduction of cortisol and aldosterone biosynthesis.

The steroid 21-hydroxylase gene, located in the HLA class III gene region on chromosome 6, has a complicated structure with a high degree of variability. An active gene, CYP21B, and a highly homologous inactive pseudogene, CYP21A, are located 3′ from each of the two genes encoding the fourth component of complement C4A and C4B, forming a repeated tandem of the units C4/21OH.

In humans, congenital adrenal hyperplasia is observed in a rather high frequency (=1 in 10,000 births) and, thus, is one of the most common inborn errors of metabolism. Affected infants die from salt loss, hyperkalemic acidosis and dehydration unless treated with steroid hormone replacement. XY genetic male patients are born with female external genitalia due to the absence of testicular testosterone synthesis.

For the diagnosis of such disease, conventionally, the method for detecting the amount of 17-OH progesterone, a substrate of 21-hydroxylase, in serum had been used. Recently, studies using oligonucleotide probes specific for CYP21A and CYP21B, southern blot/restriction fragment length polymorphism (RFLP), PCR (Polymerase Chain Reaction)/RFLP or PCR/oligonucleotide probes and sequence analysis of these genes revealed that point mutations, gene deletion, and specific gene to pseudogene conversions are the abnormalities that most often affect the CYP21B alleles in patients with congenital adrenal hyperplasia. Most of these methods require the use of molecular hybridization and some are too labor intensive for routine clinical diagnosis.

Such diagnostic technologies have been disclosed in U.S. Pat. Nos. 5,543,294; 5,807,678; 5,872,230. U.S. Pat. No. 5,543,294 disclosed a method of typing a human papillomavirus in a patient infected by a human papillomavirus. U.S. Pat. No. 5,807,678 disclosed a method for diagnosis congenital lipoid adrenal hyperplasia and for the detection of the presence of a mutated gene for steroidogenesis acute regulatory protein (StAR) by analyzing nucleic acid obtained from a patient. U.S. Pat. No. 5,872,230 disclosed a composition and methods relating to the regulation of transport of cholesterol into the mitochondria of a cell and, for the regulation of steroidogenesis.

Previously, the inventor had already developed the simple kit for diagnosing congenital adrenal hyperplasia in high accuracy, which is disclosed in WO 00/63431. However, in the present invention, the inventor improves the diagnostic kit using oligonucleotide chip.

### SUMMARY OF THE INVENTION

Thorough and intensive research repeated by the inventor aims to developing effective diagnosis for congenital adrenal hyperplasia, resulted in the finding that oligonucleotide chip containing fragments of 21-hydroxylase gene can be utilized in accurately and simply diagnosing a congenital adrenal hyperplasia through reverse dot hybridization or polymerase chain reaction-microplate hybridization.

Accordingly, it is an object of the present invention to overcome the problems encountered in prior arts and to provide a diagnostic method for congenital adrenal hyperplasia, which is useful as a routine screening assay with high accuracy in hospitals.

It is another object of the present invention to provide a diagnostic kit for congenital adrenal hyperplasia, with which the affection with and the severity of diseases can be accurately diagnosed.

In accordance with an aspect of the present invention, there is provided a diagnostic method for congenital adrenal hyperplasia, comprising the steps of: preparing oligonucleotide chip in which 7 sets of tandem cDNA fragments of normal and patient's 21-hydroxylase gene (exon 1, intron 2, exon 4) are immobilized to solid support; amplifying the DNA segments of 21-hydroxylase gene (exon 1, intron 2, exon 4) from the genomic DNA of a testee using labeled primers under the suitable condition for carrying out polymerase chain reaction (PCR) ; hybridizing 7 sets of tandem cDNA fragments in oligonucleotide chip with the PCR product by amplifying gDNA segments; and analyzing the results of the hybridization.

For preparing above oligonucleotide chip, following methods are required; i ) preparing 7 sets of constructed vectors pCAH-I2N (KCTC 0759BP), pCAH-I2N2 (KCTC 0760BP), pCAH-I2P (KCTC 0761BP), pCAH-E1N (KCTC 0758BP), pCAH-E1P (KCTC 0592BP), pCAH-E4N (KCTC 0762BP), pCAH-E4P (KCTC 0763BP), wherein said plasmid vector pCAH-I2N comprises 15 tandem copies of double stranded cDNA which sequences are [SEQ ID NO: 12] for sense insert, [SEQ ID NO: 13] for antisense insert: said plasmid vector pCAH-I2N2 comprises 23 tandem copies of double stranded cDNA which sequences are [SEQ ID NO: 14] for sense insert, [SEQ ID NO: 15] for antisense insert: said plasmid vector pCAH-I2P comprises 25 tandem copies of double stranded cDNA which sequences are [SEQ ID NO: 16] for sense insert, [SEQ ID NO: 17] for antisense insert: said plasmid vector pCAH-E1N comprises 7 tandem copies of double stranded cDNA which sequences are [SEQ ID NO: 18] for sense insert, [SEQ ID NO: 19] for anfisense insert: said plasmid vector pCAH-E1P comprises 10 tandem copies of double stranded cDNA which sequences are [SEQ ID NO: 20] for sense insert, [SEQ ID NO: 21] for antisense insert: said plasmid vector pCAH-E4N comprises 12 tandem copies of double stranded cDNA which sequences are [SEQ ID NO: 22] for sense insert, [SEQ ID NO: 23] for antisense insert: said plasmid vector pCAH-E4P comprises 19 tandem copies of double stranded cDNA which sequences are [SEQ ID NO: 24] for sense insert, [SEQ ID NO: 25] for antisense insert; ii) amplifying the tandem cDNA fragments in said 7 constructed vectors using functional group labelled primers of [SEQ ID NO: 26] and [SEQ ID NO: 27] under PCR conditions; iii) immobilizing 7 sets of amplified tandem cDNA fragments to solid support by coupling reaction for immobilization.

In accordance with another aspect of the present invention, there is provided a diagnostic kit for congenital adrenal hyperplasia, comprising: oligonucleotide chip in which 7 sets of tandem cDNA fragments of normal and patient's 21-hydroxylase gene (exon 1, intron 2, exon 4) are immobilized to solid support; primers with detectable label, which are used to amplify DNA segments from the genomic DNA of a testee; a buffer and a polymerase for polymerase chain reaction; and a buffer for gene hybridization.

### BRIEF DESCRIPTION OF DRAWINGS

FIG. 1 shows the tandemly ligated seven vectors of the present invention.

### DETAILED DESCRIPTION OF THE INVENTION

This invention provides a method of diagnosing congenital adrenal hyperplasia in a human subject which comprises i) obtaining genome DNA-containing samples from the subject; ii) amplifying three discrete portions of the subject's 21-hydroxylase gene using sets of primers where each such discrete portion containing a sequence that differentiates a wild-type allele from a corresponding pseudogene allele; iii) preparing oligonucleotide chip in which 7 sets of tandem cDNA fragments of normal and patient's 21-hydroxylase gene (exon 1, intron 2, exon 4) are immobilized to solid support for hybridizing amplified gene fragments; and iv) hybridizing the three resulting amplified discrete portions of the gene with tandem cDNA in oligonucleotide chip so as to separately determine whether each such portion contains a sequence associated with the corresponding pseudogene allele in the presence of such a sequence indicating the subject.

1) Amplification of Genome DNA

For the purposes of this invention, amplification means any method of amplification well known to those of skill in the art. Such methods of amplification include, for example, enzymatic amplification and amplification using conventional cloning techniques well known to those of skill in the art. In one embodiment of the invention, the amplification is effected by enzymatic amplification, e.g., by means of the polymerase chain reaction using sets of primers. The method of polymerase chain reaction is well known to those of skill in the art.

Genome 21-hydroxylase gene has 2880-bp [SEQ ID NO: 1] and in most cases, the mutations occur in exon 1 (P30L), intron 2 (A/C655G) and exon 4 (I172N). Specifically, in exon 1, the mutation occurs by replacing 212th base from C to T. Therefore, the 30th amino acid (Pro) is replaced by Leu, which causes the inactivation of 21-hydroxylase gene. In intron 2, the mutation occurs by replacing 665th base from A or C to G, which causes the splicing mutation to result in the truncated protein. In exon 4, the mutation occurs by replacing 815th base from T to A. Therefore, the 172th amino acid (Arg) is replaced by Trp, which causes the inactivation of 21-hydroxylase gene.

To identify the point mutation of gene, PCR amplification can be carried out. To detect the point mutation of exon 1, two steps of PCR amplification are required. In the first step, using two primers; PCR primer P1 [SEQ ID NO: 2] and PCR primer P2 [SEQ ID NO: 3], exon 1 and intron 2 of 21-hydroxylase gene is amplified. In the second step, using two primers; PCR primer E1TS [SEQ ID NO: 4] and PCR primer E1TAS [SEQ ID NO: 5], the point mutation of exon 1 (P30 L) is amplified.

To detect the point mutation of intron 2, the first step of PCR amplification is same as the step of exon 1. Then, using two primers; PCR primer I2TS [SEQ ID NO: 6] and PCR primer I2TAS [SEQ ID NO: 7], the point mutation of intron 2 (A/C655G) is amplified.

To detect the point mutation of exon 4, the first step of PCR amplification is carried out using two primers; PCR primer P3 [SEQ ID NO: 8] and PCR primer P4 [SEQ ID NO: 9]. Then, using two primers; PCR primer E4TS [SEQ ID NO: 10] and PCR primer E4TAS [SEQ ID NO: 11], the point mutation of exon 4 (1172N) is amplified.

In each primer, ³²P or fluorescent label is attached to detect the hybridization between PCR product and tandem cDNA in oligonucleotide chip. Fluorescent hybridization images of oligonucleoetide chip can be monitored with a fluorescence microscope equipped with a CCD camera and software.

2) Hybridization

The present invention pertains the diagnosis of congenital adrenal hyperplasia. For this purpose, oligonucleotide chip can be employed.

In this invention, for preparing oligonucleotide chip, the inventor designed seven sets of constructed vectors [pCAH-I2N, pCAH-I2N2, pCAH-I2P, pCAH-E1N, pCAH-E1P, pCAH-E4N, pCAH-E4P]. Detailed structures of vectors are as follows.

The vector, pCAH-I2N, is constructed by inserting 15 tandem copies of double stranded cDNA which sequences are [SEQ ID NO: 12] for sense insert, [SEQ ID NO: 13] for antisense insert, which has to be hybridized with intron 2 gene of 21-hydroxylase of normal subject.

The vector, pCAH-I2N2, is constructed by inserting 23 tandem copies of double stranded cDNA which sequences are [SEQ ID NO: 14] for sense insert, [SEQ ID NO: 15] for antisense insert, which has to be hybridized with intron 2 gene of 21-hydroxylase of normal subject.

The vector, pCAH-I2P, is constructed by inserting 25 tandem copies of double stranded cDNA which sequences are [SEQ ID NO: 16] for sense insert, [SEQ ID NO: 17] for antisense insert, which has to be hybridized with intron 2 gene of 21-hydroxylase of patient.

The vector, pCAH-E1N, is constructed by inserting 7 tandem copies of double stranded cDNA which sequences are [SEQ ID NO: 18] for sense insert, [SEQ ID NO: 19] for antisense insert, which has to be hybridized with exon 1 gene of 21-hydroxylase of normal subject.

The vector, pCAH-E1P, is constructed by inserting 10 tandem copies of double stranded cDNA which sequences are [SEQ ID NO: 20] for sense insert, [SEQ ID NO: 21] for antisense insert, which has to be hybridized with exon 1 gene of 21-hydroxylase of patient.

The vector, pCAH-E4N, is constructed by inserting 12 tandem copies of double stranded cDNA which sequences are [SEQ ID NO: 22] for sense insert, [SEQ ID NO: 23] for antisense insert, which has to be hybridized with exon 4 gene of 21-hydroxylase of normal subject.

The vector, pCAH-E4P, is constructed by inserting 19 tandem copies of double stranded cDNA which sequences are [SEQ ID NO: 24] for sense insert, [SEQ ID NO: 25] for antisense insert, which has to be hybridized with exon 4 gene of 21-hydroxylase of patient.

The constructed vectors were deposited on Korean Collection for Type Cultures of Korean Research Institute of Bioscience and Biotechnology #52, Oun-dong, Yusong-ku, Taejon 305-333, Republic of Korea under Budapest Treaty. The deposition numbers and dates of each vectors are as follows.

pCAH-I2N was deposited under deposition No. KCTC 0759BP on Mar. 23, 2000. pCAH-I2N2 was deposited under deposition No. KCTC 0760BP on Mar. 23, 2000. pCAH-I2P was deposited under deposition No. KCTC 0761BP on Mar. 23, 2000. pCAH-E1N was deposited under deposition No. KCTC 0758BP on Mar. 23, 2000. pCAH-E1P was deposited under deposition No. KCTC 0592BP on Mar. 19, 1999. pCAH-E4N was deposited under deposition No. KCTC 0762BP on Mar. 23, 2000. pCAH-E4P was deposited under deposition No. KCTC 0763BP on Mar. 23, 2000.

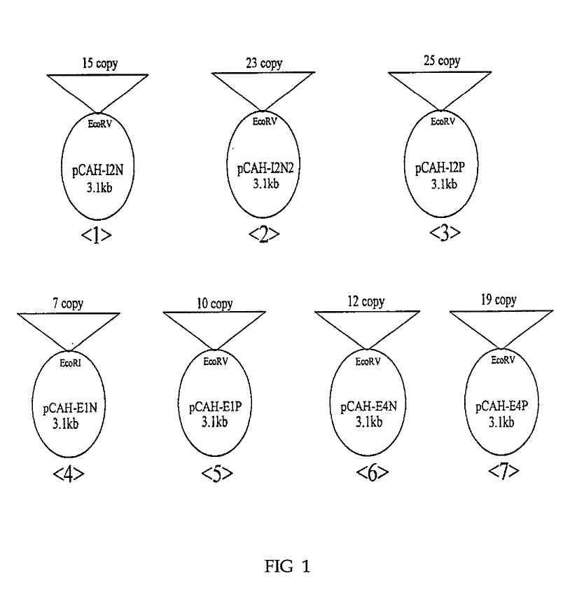

FIG. 1 shows the tandemly ligated seven vectors. The cDNA inserts are tandemly constructed in unidirection at SfiI restriction sites of plasmid vectors pBluescript-KS (2961 bp) sold by Stratagene.

Then, the tandem cDAN fragments in above 7 constructed vectors are amplified by PCR using functional group labelled primers of 5′-aattaaccct cactaaaggg-3′ [SEQ ID NO: 26] and 5′-gtaatacgac tcactatagg gc-3′ [SEQ ID NO: 27]. Primers used for PCR amplification are specific to vector. Therefore, all primers used for each 7 constructed vectors are same. Further, the nucleophilic functional group consisting of alcohol, amine or thiol has to be labelled at the end of sequence of primers in order to immobilize amplified cDNA fragments to solid support by coupling reaction through the bond consisting of ester, amide or thioester between functional group of cDNA fragment and functional group of solid support. Finally, after removing the unimmobilized cDNA fragments, the oligonucleotide chip has prepared.

In normal subject, the hybridization between cDNA fragments from following 4 vectors, pCAH-I2N, pCAH-I2N2, pCAH-E1N, pCAH-E4N, in oligonucleotide chip and amplified gDNA fragments shall be performed, because these cDNA fragments are hybridized with normal exon 1, intron 2 and exon 4 of 21-hydroxylase gene.

In patient, the hybridization between cDNA fragments from following 3 vectors, pCAH-I2P, pCAH-E1P, pCAH-E4P, in oligonucleotide chip and amplified gDNA fragments shall be performed, because these cDNA fragments are hybridized with point mutation of exon 1, intron 2 and exon 4 of 21-hydroxylase gene. The hybrids are monitored with a fluorescence microscope equipped with a CCD camera and software.

Intensive signals are detected by the hybridization at cDNA fragments from vectors pCAH-I2N, pCAH-I2N2, pCAH-E1N, pCAH-E4N for normal subject, whereas, other intensive signals are detected by the hybridization at cDNA fragments from vectors pCAH-I2P, pCAH-E1P, pCAH-E4P for CAH patient.

The PCR template may be obtained from the blood of the individual to be diagnosed. Genomic DNA is isolated from the blood and subjected to PCR to amplify the segment of interest. This DNA segment is added to oligonucleotide chip where fragments of 21-hydroxylase gene are immobilized. If intensive signals are detected at the cDNA fragments from vectors pCAH-I2N, pCAH-I2N2, pCAH-E1N, pCAH-E4N in oligonucleotide chip, the individual is normal. On the other hand, if intensive signals are detected at the cDNA fragments from vectors pCAH-I2P, pCAH-E1P, pCAH-E4P in oligonucleotide chip, the subject is a patient.

A better understanding of the present invention may be obtained in light of the following examples which are set forth to illustrate, but are not to be construed to limit the present invention.

### EXAMPLES

### Example 1

### Polymerase Chain Reaction

Genome DNA was isolated from the peripheral blood of the subject and used as template in PCR to amplify the 21-hydroxylase gene. As the primers for this FCR, several oligonucleotides were constructed.

Primers used for each part of genome DNA were as follows.

i) For exon 1, two steps of PCR amplification were required, those were, in the first step, required for two primers; PCR primer P1 [SEQ ID NO: 2] and PCR primer P2 [SEQ ID NO: 3], in the second step, required for biotin-labeled two primers; PCR primer E1TS [SEQ ID NO: 4] and PCR primer E1TAS [SEQ ID NO: 5], for amplifying the point mutation of exon 1 (P30 L).

ii) For intron 2, two steps of PCR amplification were required, those were, in the first step, required for two primers; PCR primer P1 [SEQ ID NO: 2] and PCR primer P2 [SEQ ID NO: 3], in the second step, required for biotin-labeled two primers; PCR primer I2TS [SEQ ID NO: 6] and PCR primer I2TAS [SEQ ID NO: 7], for amplifying the point mutation of intron 2 (A/C655G).

iii) For exon 4, two steps of PCR amplification were required, those were, in the first step, required for two primers; PCR primer P3 [SEQ ID NO: 8] and PCR primer P4 [SEQ ID NO: 9], in the second step, required for biotin-labeled two primers; PCR primer E4TS [SEQ ID NO: 10] and PCR primer E4TAS [SEQ ID NO 11], for amplifying the point mutation of exon 4 (I172N).

The specific conditions for amplification of genome DNA were as follows.

Amplification for Exon 1

i) For distinguishing true gene and pseudo gene, CAH gene was amplified using two primers SEQ ID NO: 2 and SEQ ID NO: 3. 100 ng of genomic DNA, 10 pmole of fluorescent labelled two primers, 4 , μL of 2.5 mM dNTP and Taq polymerase (10 unit) were added with buffer solution (20 mM of Tris-HCl pH 8.0, 10 mM of potassium chloride, 0.1 mM of EDTA) to make a final volume of 50 μL. This PCR mixture was denaturated at 94° C. for 5 minutes. The PCR consisted of 35 thermal cycles in each of a denaturing process at 94° C. for 30 sec, annealing processes at 60° C. for 30 sec, at 72° C. for 2 minutes. First PCR product obtained was 1339 bp.

ii) In the second step, first PCR product was diluted at 10 times. 1 μL of 1/10 diluted first PCR product, 4 μL of 2.5 mM dNTP and 10 pmole of fluorescent labelled primers SEQ ID NO: 4 and SEQ ID NO: 5. Tag polymerase (10 unit) were added with buffer solution (20 mM of Tris-HCl pH 8.0, 10 mM of potassium chloride, 0.1 mM of EDTA) to make a final volume of 50 μL. This PCR mixture was denaturated at 94° C. for 5 minutes. The PCR consisted of 30 thermal cycles in each of a denaturing process at 94° C. for 30 sec, annealing processes at 58° C. for 30 sec, at 72° C. for 1 minute. Final PCR product was obtained.

Amplification for Intron 2 and Exon 4

i ) For intron 2, the two step PCR amplification were carried out in the same manners described for exon 1 except that the fluorescent labelled primers used in the second step were SEQ ID NO: 6 and SEQ ID NO: 7. The final PCR product was 104 bp.

ii) For exon 4, the two step PCR amplification were carried out in the same manners described for exon 1 except that the fluorescent labelled primers used in the first step were SEQ ID NO: 8 and SEQ ID NO: 9, and in the second step SEQ ID NO: 10 and SEQ ID NO: 11, and that the annealing process at 72° C. for 10 minutes.

### Example 2

### Construction of Vectors

As preferred substrates for hybridization, the inventors designed seven sets of constructed vectors [pCAH-I2N, pCAH-I2N2, pCAH-I2P, pCAH-E1N, pCAH-E1P, pCAH-E4N, pCAH-E4P]. Detailed structures of vectors are as follows.

The vector, pCAH-I2N, is constructed by inserting 15 tandem copies of double stranded cDNA which sequences are [SEQ ID NO: 12] for sense insert, [SEQ ID NO: 13] for antisense insert, which has to be hybridized with intron 2 gene of 21-hydroxylase of normal subject.

The vector, pCAH-I2N2, is constructed by inserting 23 tandem copies of double stranded cDNA which sequences are [SEQ ID NO: 14] for sense insert, [SEQ ID NO: 15] for antisense insert, which has to be hybridized with intron 2 gene of 21-hydroxylase of normal subject.

The vector, pCAH-I2P, is constructed by inserting 25 tandem copies of double stranded cDNA which sequences are [SEQ ID NO: 16] for sense insert, [SEQ ID NO: 17] for antisense insert, which has to be hybridized with intron 2 gene of 21-hydroxylase of patient.

The vector, pCAH-E1N, is constructed by inserting 7 tandem copies of double stranded cDNA which sequences are [SEQ ID NO: 18] for sense insert, [SEQ ID NO: 19] for antisense insert, which has to be hybridized with exon 1 gene of 21-hydroxylase of normal subject.

The vector, pCAH-E1P, is constructed by inserting 10 tandem copies of double stranded cDNA which sequences are [SEQ ID NO: 20] for sense insert, [SEQ ID NO: 21] for antisense insert, which has to be hybridized with exon 1 gene of 21-hydroxylase of patient.

The vector, pCAH-E4N, is constructed by inserting 12 tandem copies of double stranded cDNA which sequences are [SEQ ID NO: 22] for sense insert, [SEQ ID NO: 23] for antisense insert, which has to be hybridized with exon 4 gene of 21-hydroxylase of normal subject.

The vector, pCAH-E4P, is constructed by inserting 19 tandem copies of double stranded cDNA which sequences are [SEQ ID NO: 24] for sense insert, [SEQ ID NO: 25] for antisense insert, which has to be hybridized with exon 4 gene of 21-hydroxylase of patient.

The construction of vectors were prepared as following steps:

i) pBluescript-KSII [(2.96 kb) Stratagene] was used as backbone of vector and this vector was digested using restriction enzyme EcoRV at 37° C. for 3 hours to make blunt-end status. After purification, the vector was treated with alkaline phosphatase (TAKARA) at 37° C. for 1 hour to be dephosphorylated. Then, obtained vector was stored at 20° C. after purification.

ii) Ligation was carried out to insert the copies of DNA inserts, which was appropriate for assembly of vector to be described above. The ligation was carried out using T4 DNA ligase (10 unit) at 15° C. for 1˜2 days.

### Example 3

### Oligonucleotide Chip

The tandem cDAN fragments in above 7 constructed vectors are amplified by PCR using functional group labelled primers of 5′-aattaaccct cactaaaggg-3′ [SEQ ID NO: 26] and 5′-gtaatacgac tcactatagg gc-3′ [SEQ ID NO: 27]. Primers used for PCR amplification are specific to vector.

200 ng of template (plasmid), 20 pmole of two functional group labelled primers, 4 μL of 2.5 mM dNTP and 1 μL of Taq polymerase (5 unit/ μL) were added with buffer solution (20 mM of Tris-HCl pH 8.0, 10 mM of potassium chloride, 0.1 mM of EDTA) to make a final volume of 49 μL in 0.2 ml tube. This mixture tube was transferred to thermocycler. This mixture was denaturated at 94° C. for 5 minutes. The PCR consisted of 35 thermal cycles in each of a denaturing process at 94° C. for 1 min, annealing processes at 60° C. for 1 min. Then, the PCR product was held at 72° C. for additional 10 min, and it was cooled to 4° C. Finally, functional group labelled PCR product was obtained.

Then, labelled amplified cDNA fragments are immobilized to solid support by coupling reaction through the bond consisting of ester, amide or thioester between functional group of cDNA fragment and functional group of solid support. Finally, after removing the unimmobilized cDNA fragments, the oligonucleotide chip has prepared. As solid support, polystyrene, silica gel, polyacrylamide, polyacrylate, polyamide, hydroxyethyl or methacrylate can be used.

### Example 4

### Oligonucleotide Chip Hybridization

On the oligonucleotide chip, the genomic DNAs of testee was added, after performing PCRs using primers as described in example 1 whose 5′-ends were labeled with fluorescence. The PCR products thus obtained were subjected to hybridization. Subsequently, the oligonucleotide chip was added with hybridization buffer (2.5×SSC). After hybridization between the PCR products and cDNA fragments on the oligonucleotide chip, fluorescent hybridization images of oligonucleoetide chip can be monitored with a fluorescence microscope equipped with a CCD camera and software.

Followings are detailed procedures of oligonucleotide chip hybridization.

i) Spotting

5′-amino-linker was covalently attached to oligonucleotide by modification of the oligonucleotides directly during oligonucleotide synthesis. Then, the oligonucleotide was resuspended in distilled water at the desired concentration (100 mol/μL). The amino-modified oligonucleotide was transferred into 384 well plates. The oligonucleotide was printed onto superaldehyde coating slides by placing the 384 well plates using Biorobotics spotter. After printing, the slides was left at room temperature for 24 hours to permit for drying the DNA onto the surface of the silylated slides.

ii) Spotted slide washing

The slide was rinsed twice in 0.2% of SDS for 2 min at room temperature. The slide was rinsed twice with distilled water for 2 min. The slide was transferred into distilled water at 100° C. for 2 min. Then, it was cooled at room temperature for 5 min. After dissolving 0.65 g of sodium borohydride (NaBH4) in 187.5 ml of PBS, 62.5 ml of 100% ethanol was added (prepare just prior to use for reducing bubbling) at room temperature. The slide was rinsed three times in 0.2% of SDS for 1 min. Then, the slide was rinsed once with distilled water for 1 min at room temperature. Finally, the slide was dried by centrifugation for 1 min at 500×g

iii) Hybridization

Target mixture contained pre-denatured product (about 100 ng) and hybridization buffer (5×SSC). Total volume was 12 μL, and hybridization was preformed at 42° C. for 3 hours.

iv) Washing

The slide was washed in 0.2% of SDS and 2×SSC for 15 min at room temperature. Then, the slide was washed again in distilled water for 1 min at room temperature, followed by washing in 2×SSC for 5 min. Finally, the slide was dried by centrifugation.

v) Scanning

Scanning was imaged on the GSI ScanArray.

In normal subject, the hybridization between cDNA fragments from following 4 vectors, pCAH-I2N, pCAH-I2N2, pCAH-E1N, pCAH-E4N, in oligonucleotide chip and amplified gDNA fragments shall be performed, because these cDNA fragments are hybridized with normal exon 1, intron 2 and exon 4 of 21-hydroxylase gene.

In patient, the hybridization between cDNA fragments from following 3 vectors, pCAH-I2P, pCAH-E1P, pCAH-E4P, in oligonucleotide chip and amplified gDNA fragments shall be performed, because these cDNA fragments are hybridized with point mutation of exon 1, intron 2 and exon 4 of 21-hydroxylase gene. The hybrids are monitored with a fluorescence microscope equipped with a CCD camera and software.

Intensive signals are detected by the hybridization at cDNA fragments from vectors pCAH-I2N, pCAH-I2N2, pCAH-E1N, pCAH-E4N for normal subject, whereas, other intensive signals are detected by the hybridization at cDNA fragments from vectors pCAH-I2P, pCAH-E1P, pCAH-E4P for CAH patient.

What is claimed is:

1. A diagnostic method for congenital adrenal hyperplasia, comprising the steps of:
i) preparing oligonucleotide chip in which 7 sets of tandem cDNA fragments of normal and patient's 21-hydroxylase gene (exon 1, intron 2, exon 4) are immobilized to solid support;
ii) amplifying the DNA segments of 21-hydroxylase gene (exon 1, intron 2, exon 4) from the genomic DNA of a testee using labeled primers under the suitable condition for carrying out polymerase chain reaction (PCR);
iii) hybridizing 7 sets of tandem cDNA fragments in oligonucleotide chip with the PCR product by amplifying gDNA segments; and
iv) analyzing the results of the hybridization.

2. The diagnostic method for congenital adrenal hyperplasia according to claim 1, wherein said oligonucleotide chip is prepared by following method comprising the steps of:
i) preparing 7 sets of constructed vectors pCAH-I2N (KCTC 0759BP), pCAH-I2N2 (KCTC 0760BP), pCAH-I2P (KCTC 0761BP), pCAH-E1N (KCTC 0758BP), pCAH-E1P (KCTC 0592BP), pCAH-E4N (KCTC 0762BP), pCAH-E4P (KCTC 0763BP);
ii) amplifying the tandem cDNA fragments in said 7 constructed vectors using functional group labelled primers of [SEQ ID NO: 26] and [SEQ ID NO: 27] under PCR conditions; and
iii) immobilizing 7 sets of amplified tandem cDNA fragments to solid support by coupling reaction for immobilization.

3. The diagnostic method for congenital adrenal hyperplasia according to claim 2, wherein said plasmid vector pCAH-I2N comprises 15 tandem copies of double stranded cDNA which sequences are [SEQ ID NO: 12] for sense insert, [SEQ ID NO: 13] for antisense insert

4. The diagnostic method for congenital adrenal hyperplasia according to claim 2, wherein said plasmid vector pCAH-I2N2 comprises 23 tandem copies of double stranded cDNA which sequences are [SEQ ID NO: 14] for sense insert, [SEQ ID NO: 15] for antisense insert

5. The diagnostic method for congenital adrenal hyperplasia according to claim 2, wherein said plasmid vector pCAH-I2P comprises 25 tandem copies of double stranded cDNA which sequences are [SEQ ID NO: 16] for sense insert, [SEQ ID NO: 17] for antisense insert

6. The diagnostic method for congenital adrenal hyperplasia according to claim 2, wherein said plasmid vector pCAH-E1N comprises 7 tandem copies of double stranded cDNA which sequences are [SEQ ID NO: 18] for sense insert, [SEQ ID NO: 19] for antisense insert

7. The diagnostic method for congenital adrenal hyperplasia according to claim 2, wherein said plasmid vector pCAH-E1P comprises 10 tandem copies of double stranded cDNA which sequences are [SEQ ID NO: 20] for sense insert, [SEQ ID NO: 21] for antisense insert

8. The diagnostic method for congenital adrenal hyperplasia according to claim 2, wherein said plasmid vector pCAH-E4N comprises 12 tandem copies of double stranded cDNA which sequences are [SEQ ID NO: 22] for sense insert, [SEQ ID NO: 23] for antisense insert

9. The diagnostic method for congenital adrenal hyperplasia according to claim 2, wherein said plasmid vector pCAH-E4P comprises 19 tandem copies of double stranded cDNA which sequences are [SEQ ID NO: 24] for sense insert, [SEQ ID NO: 25] for antisense insert

10. A diagnostic kit for congenital adrenal hyperplasia, comprising:
i ) oligonucleotide chip in which 7 sets of tandem cDNA fragments of normal and patient's 21-hydroxylase gene (exon 1, intron 2, exon 4) are immobilized to solid support;
ii) primers with detectable label, which are used to amplify DNA segments from the genomic DNA of a testee;
iii) a buffer and a polymerase for polymerase chain reaction; and
iv) a buffer for gene hybridization.

11. The diagnostic kit according to claim 10, wherein said detectable label comprises ³²P radiolabel or fluorescence-label.
